(19) United States
(12) Patent Application Publication
(10) Pub. No.: US 2003/0120192 A1
(43) Pub. Date: Jun. 26, 2003

(54) Human body traction and mending apparatus

(76) Inventor: Richard C. C. Chao, Taipei (TW)

Correspondence Address: BACON & THOMAS, PLLC, 625 SLATERS LANE, FOURTH FLOOR, ALEXANDRIA, VA, 22314

(21) Appl. No.: 10/022,199

(22) Filed: Dec. 20, 2001

(51) Int. Cl. A61F 5/00

(52) U.S. Cl. 602/36; 602/32

(57) ABSTRACT

A human body traction and mending apparatus consists of an inflation member, a control means mounted to the pouch, a delivery means connecting to the control means, and a fluid generation means for coupling to a human hand, leg, waist or neck to support or stretch a patient's sore body portion for achieving healing and mending purpose. The traction and mending apparatus of the invention is portable and may be worn on human's hand, leg, waist or neck for carrying outdoors.

### FIELD OF THE INVENTION

The present invention relates to a human body traction and mending apparatus and particularly a traction and mending apparatus for use on a human hand, leg, waist and neck, and is portable to allow a patient to carry and use outdoors.

### BACKGROUND OF THE INVENTION

People who do exercises without proper physical warming usually will result in some sorts of body injury. Some times people who sleep at not proper postures also tend to inflict sore waist and neck. All this could cause inconvenience to people's life and work.

It is especially troublesome when people have injury on the cervical vertebrae. In such a case, doctors usually will put a body mending device around patient's neck to support the spine at the neck portion to prevent the cervical vertebrae from wobbling or bending, and to prevent the cervical vertebrae from incurring pain. The body mending device also helps to heal the injured cervical vertebrae.

A conventional body mending device is generally made of metal and a foam pad lining attached to the inner side of the mending device to prevent the metal from hurting the patient. The mending device thus made is quite heavy and becomes a burden to users who have to wear the mending device for a long period of time. As a result, healing is less effective and healing time is longer.

### SUMMARY OF THE INVENTION

The primary object of the invention is to resolve aforesaid disadvantages. The traction and mending apparatus of the invention allows a fluid to inject inside to support the sore portion of the patient, and to stretch and mend the sore portion, and is portable to allow the patient carrying conveniently wherever desired.

Another object of the invention is to provide a single traction, support and mending for the sore portion of the patient.

Still another object of the invention is to provide a fluid generation means to inject fluid into the traction and mending apparatus to maintain the support and traction effects of the traction and mending apparatus.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a perspective view of the invention.

FIG. 2 is an exploded view of the invention according to FIG. 1.

FIG. 3A is a cross section of the invention taking along line 3A-3A in FIG. 1.

FIG. 3B is a fragmentary enlarged view of FIG. 3A.

FIG. 4 is a schematic view of another embodiment of the invention.

FIG. 5 is a schematic view of yet another embodiment of the invention.

FIG. 6A is a schematic view of still another embodiment of the invention.

FIG. 6B is a front view of a connection head according to FIG. 6A.

FIG. 7 is a pictorial view of the invention in use.

FIG. 8 is another pictorial view of the invention in use.

### DESCRIPTION OF THE PREFERRED EMBODIMENTS

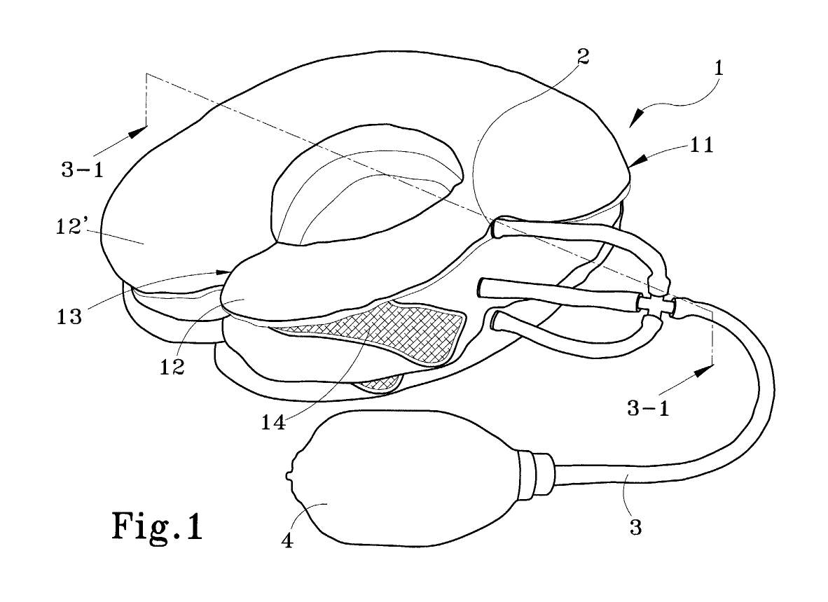

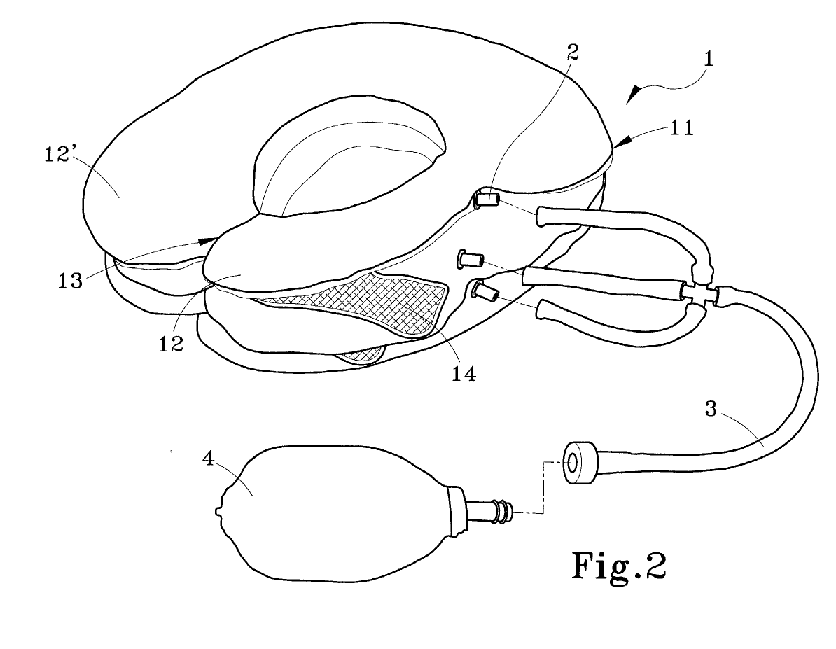

Referring to FIGS. 1 and 2, the human body traction and mending apparatus of the invention consists of an inflation member 1, a control means 2 mounted to a selected location of the inflation member 1, a delivery means 3 connecting to the control means 2, and a fluid generation means 4. The apparatus may be worn on the hand, foot, waist or neck of human body to support the sore portion of a patient for mending purpose, and is convenient for the patient to carry outdoors.

The inflation member 1 includes at least one pouch 11 for containing fluid (gas). The pouch 11 may fluidly communicate or not communicate with each other. The pouch 11 has two free ends 12, 12′ to form a slot opening 13 therebetween to allow human hand, foot, waist or neck to pass through. The free ends 12, 12′ have respectively a fasten element 14 attached thereon to allow the inflation member 1 coupling securely to the human hand, foot, waist or neck. The fasten element 14 may be a Velcro strip, adhesive tape, button, or zipper.

The control means 2 may be, but not limit to, a check valve mounting to the inflation member 1 or between the delivery means 3 and the fluid generation means 4. The delivery means 3 includes an inlet 21 and an outlet 22 for the fluid to pass through, and a valve stem 23 located therein movable by the fluid to regulate or restrict fluid flow, and an elastic element 24. At one end of the valve stem 23, there is a rod 25 for moving the valve stem 23.

The delivery means 3 is a pliable tubular element to allow the fluid to flow therein and has one end connecting to the control means 2 and another end connecting to the fluid generation means 4.

The fluid generation means 4 is a pliable air inflation bulb that is inflatable and deflatable. When the fluid generation means 4 is under an external compression force, an air fluid will be generated to flow into the interior of the inflation member 1 to expand and inflate the inflation member 1 so that the inflation member 1 can be used to support human hand, foot, waist or neck at a mending posture.

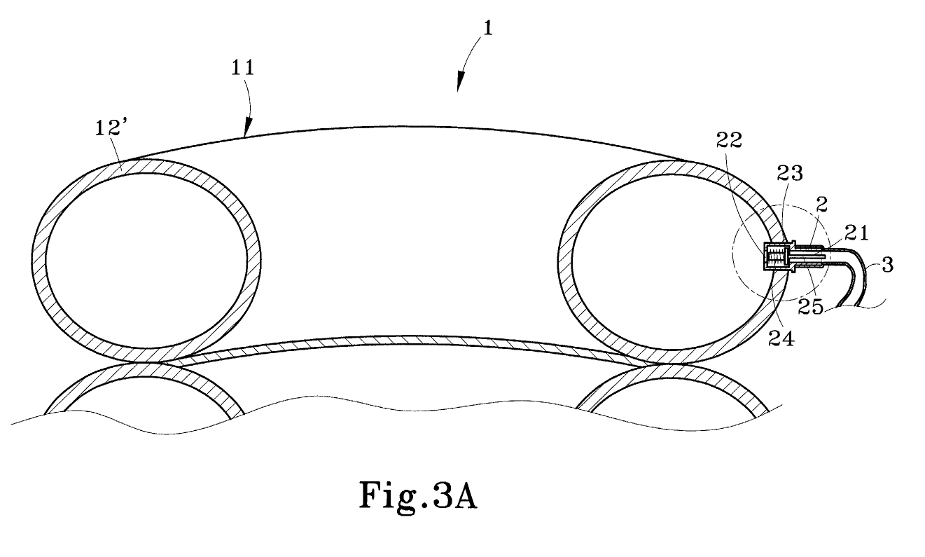

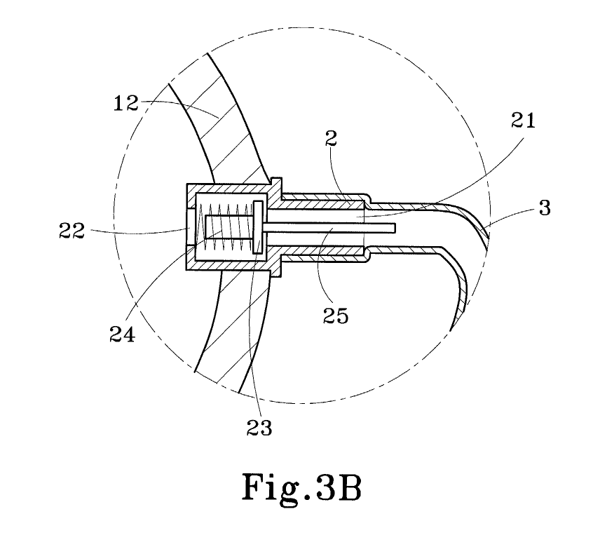

Referring to FIGS. 3A and 3B, when the fluid generation means 4 is deflated or inflated under an external force, it generates fluid and delivers to the control means 2 through the delivery means 3. The fluid pushes the valve stem 23 open and flows from the inlet 21 to the outlet 22 into every pouch 11 of the inflation member 1, and every pouch 11 is inflated and expanded.

When the fluid generation means 4 does not generate fluid for delivery, the elastic element 24 will push the valve stem 23 to its original position to prevent the fluid from escaping. Once the fluid is delivered, the patient may disengage the delivery means 3 from the control means 2 so that the patient may carry only the inflation member 1 for use outdoors or wherever desired without the delivery means 3 attached to the control means 2. Of course, the patient may also carry the delivery means 3 and fluid generation means 4 to replenish and inflate the inflation member 1 whenever needed.

In the event of the pouch 11 has been filled with too much fluid, user may push the rod 25 to move the valve stem 23 away for discharging excessive fluid and adjusting the support condition.

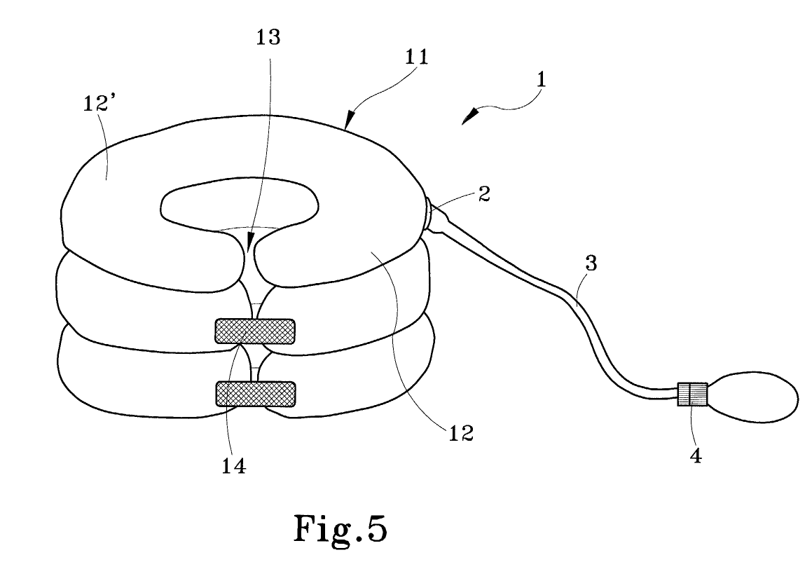

Referring to FIG. 5 for another embodiment of the invention in which the two free ends (wings) 12, 12′ of the pouch 11 of the inflation member 1 do not communicate with each other, and the free ends 12, 12′ of each pouch 11 have respectively a control means 2 located thereon. Hence the free ends 12, 12′ may be inflated or not inflated separately to adjust the elevation of the inflation member 1 at any one side to suit user's requirements. For instance, if the patient suffers a sore neck on the left side, the inflation member 1 may be inflated on the left side to lift the left side of the neck to alleviate the pain and achieve mending effect.

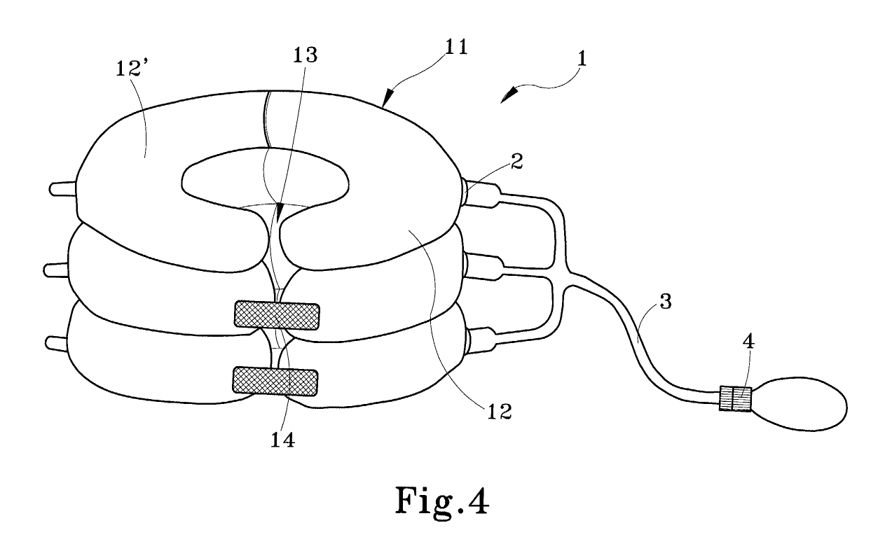

Referring to FIG. 4 for yet another embodiment of the invention, the pouch 11 of every inflation member 1 is also allowed to communicate with one another, hence only one control means 2 is required to install one of the pouches 11 to achieve inflation or deflation purpose. Such a construction is easier to produce and use.

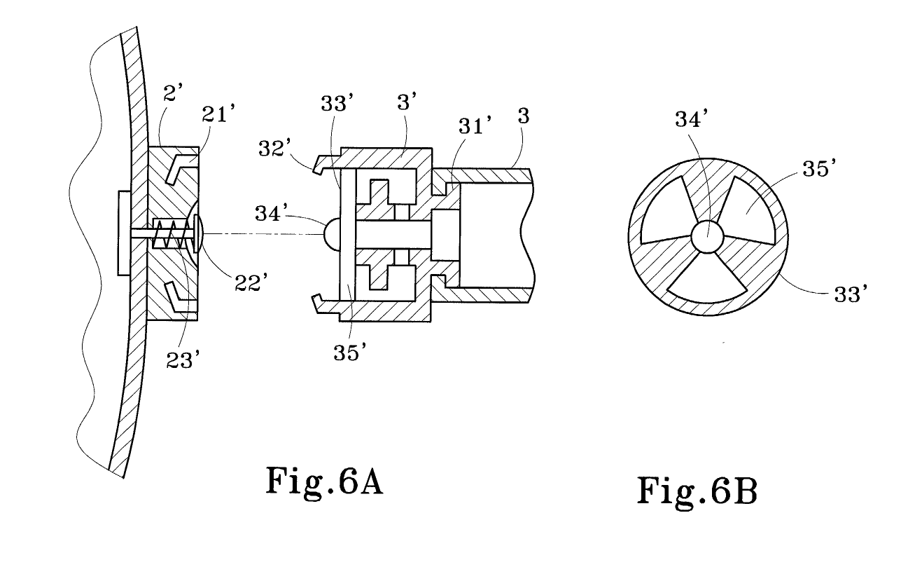

Referring to FIGS. 6A and 6B for still another embodiment of the invention, the control means 2′ has an indented recess 21′ formed in a thick end thereof, and the deliver means 3 has a connection head 3′ located at the front end thereof. The connection head 3′ further has a rear end formed a connection section 31′ to connect with the deliver means 3 and a front end formed a connection flange 32′ which is matching and engageable with the indented recess 21′. When the connection flange 32′ is engaged with the indented recess 21′ and the connection head 3′ is turned, the connection head 3′ may be coupled securely with the control means 2′. The connection head 3′ further has an inner bottom 33′ with a bulged spot 34′ formed thereon. When the connection head 3′ is coupled with the control means 2′, the bulged spot 34′ will depress a rod 22′ of the control means 2′ to make the control means 2′ open and allow fluid to flow into the pouch 11 through openings 35′ formed on the inner bottom 33′ of the connection head 3′.

When the pouch 11 is filled with fluid, the connection section 31′ may be disconnected and removed. The elastic 23′ located in the control means 2′ will push the rod 22′ to its original position to prevent the fluid from escaping. Thus complete the inflation of the pouch 11.

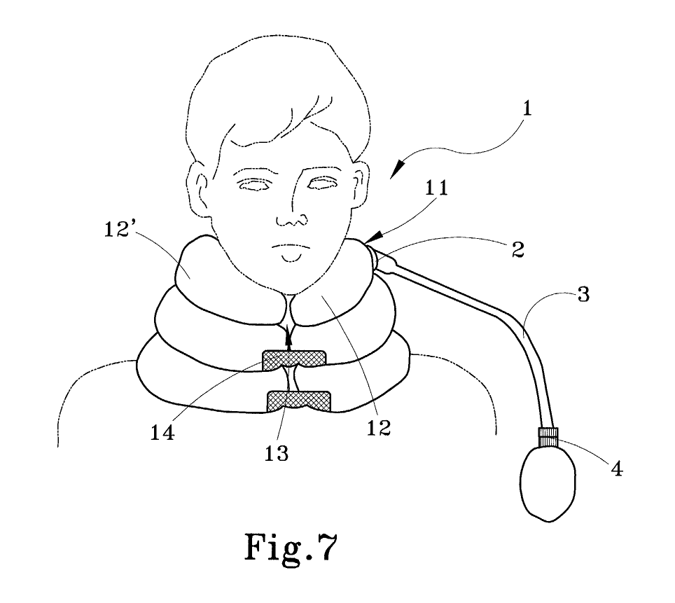

Referring to FIG. 7 for a condition of the invention in use for healing a patient's sore neck, the traction and mending apparatus of the invention is coupled to the rear side of the neck, and the two free ends 12, 12′ of the pouch 11 are fastened together by the fasten element 14 so that the inflation member 1 is anchored securely on the neck without loosening off. Then the patient may use the fluid generation means 4 to generate fluid to pump into the inflation member 1 to support or stretch patient's neck for mending and healing purpose. Such a construction also is convenient for the patient to carry around.

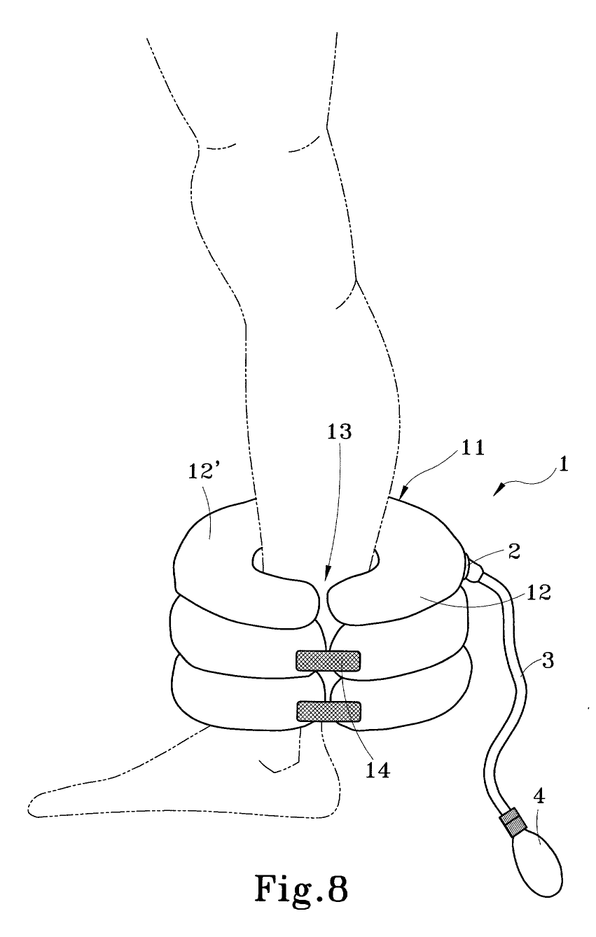

Referring to FIG. 8 for a condition of the invention in use for healing a patient's sore leg, the traction and mending apparatus of the invention is coupled to a patient's leg, and the two free ends 12, 12′ of the pouch 11 are fastened together by the fasten element 14 so that the inflation member 1 is anchored securely on the leg without loosening off. Then the patient may use the fluid generation means 4 to generate fluid to pump into the inflation member 1 to support or stretch patient's leg for mending and healing purpose. Such a construction also is convenient for the patient to carry around.

What is claimed is:

1. A human body traction and mending apparatus being portable and for supporting a sore portion of a patient's body for mending purpose, comprising:
an inflation member including at least one pouch which has two free ends with a slot opening formed therebetween, the pouch being not communicating with one another;
a control means mounting to the pouch;
a delivery means having one end connecting to the control means; and
a fluid generation means connecting to another end of the delivery means;
wherein the fluid generation means generates fluid and delivers the fluid through the delivery means into the pouch of the inflation means for inflating the pouch.

2. The human body traction and mending apparatus of claim 1, wherein the free ends have respectively attached to a fasten element, the fasten element being selected from the group consisting of Velcro strips, adhesive tapes, buttons, or zippers.

3. The human body traction and mending apparatus of claim 1, wherein the control means is a check valve located between the delivery means and the fluid generation means.

4. The human body traction and mending apparatus of claim 1, wherein the control means includes an inlet, an outlet, a valve stem and an elastic element, the valve stem having one end attached to a rod.

5. The human body traction and mending apparatus of claim 1, wherein the control means has an indented recess.

6. The human body traction and mending apparatus of claim 1, wherein the delivery means is a pliable tubular element and connects to a connection head which has a rear end formed a connection section for connecting the delivery means, a front end formed a connection flange, and an inner bottom with a bulged spot formed thereon, the inner bottom further having openings formed thereon.

7. The human body traction and mending apparatus of claim 1, wherein the fluid generation means is a pliable gas inflation bulb that is inflatable and deflatable.

8. A human body traction and mending apparatus being portable and for supporting a sore portion of a patient's body for mending purpose, comprising:
an inflation member including at least one pouch which has two free ends with a slot opening formed therebetween, the pouch being communicating with one another;
a control means mounting to the pouch;
a delivery means having one end connecting to the control means; and
a fluid generation means connecting to another end of the delivery means; wherein the fluid generation means generates fluid and delivers the fluid through the delivery means into the pouch of the inflation means for inflating the pouch.

9. A human body traction and mending apparatus being portable and for supporting a sore portion of a patient's body for mending purpose, comprising:
an inflation member including at least one pouch which has two free ends with a slot opening formed therebetween, the two free ends of each pouch being not communicating with each other;
a control means mounting to each of the two free ends of the pouch;
a delivery means having one end connecting to the control means; and
a fluid generation means connecting to another end of the delivery means; wherein the fluid generation means generates fluid and delivers the fluid through the delivery means into the pouch of the inflation means for inflating the pouch on a single side thereof as desired.

10. The human body traction and mending apparatus of claim 9, wherein the pouch communicates with one another, and the two free ends being not communicating with each other, and the control means being mounted to each of the two free ends of each pouch.
